(19) United States
(12) Patent Application Publication
(10) Pub. No.: US 2021/0249111 A1
(43) Pub. Date: Aug. 12, 2021

(54) MEDICAL PLATFORM UNIFICATION TOOL

(71) Applicant: Bhuren Patel, Needham, MA (US)

(72) Inventor: Bhuren Patel, Needham, MA (US)

(21) Appl. No.: 17/023,281

(22) Filed: Sep. 16, 2020

Related U.S. Application Data
(63) Continuation of application No. 15/968,047, filed on May 1, 2018, now abandoned.
(60) Provisional application No. 62/492,444, filed on May 1, 2017.

(51) Int. Cl. G16H 20/10; G16H 10/60; G16H 40/20

(52) U.S. Cl. CPC G16H 20/10; G16H 40/40; G16H 40/20; G16H 10/60

(57) ABSTRACT

The present invention relates to novel unified interfaces that are suitable for reducing risk of errors introduced into multi-step and multi-center medical programs through the unification of the multiple platforms of each center. In particular, the present invention provides methods, tools, and systems that unify at least three centers, e.g., of medication administration programs, reducing the number of potential errors that could be introduced, more sufficiently adhering to the five rights while utilizing and unifying the multiple platforms, for example, as needed for medical administration programs for community residential programs.

### RELATED APPLICATIONS

This application claims priority to co-pending US Utility application Ser. No. 15/968,047, filed May 1, 2018, under Attorney Docket No. MYM-001, and which claims the benefit of priority of U.S. Provisional Patent Application No. 62/492,444, filed May 1, 2017, the entire contents of which is hereby incorporated by reference herein.

### BACKGROUND OF THE INVENTION

Community residential programs that ensure the safety of individuals living within them, such as group homes, exist all over the country. These community residential programs establish standards and safeguards to protect the wellbeing of people, very often through state-wide supported efforts. The overall goal of one of these efforts is to ensure that there are appropriate policies and procedures in place for safe medication administration, i.e., helping people receive their medication while living in the community and carrying on their day to day activities. Such programs are very often referred to as medication administration programs (MAPs), and make it possible for direct care staff, who are familiar with the specific needs of each individual, to administer medication as a normal part of the person's daily routine.

In these MAPs, the direct authorization model allows trained and certified unlicensed direct care staff to administer or assist in the administration of medications to individuals living in residences in the community. Although licensed health care professionals (nurses, pharmacists, physicians) are available to help MAP certified direct care staff with any medication administration issues, an individual's prescribed medications are ordered by an authorized prescriber (physician, dentist, etc.) and dispensed by pharmacists; and then these medications are obtained from the pharmacies.

MAP certified staff are responsible for following the health care provider's instructions, which are specific to the individual and are issued by the prescribing healthcare provider (HCP), creating enumerable individualized plans that must be tracked and administered individually. Moreover, generally regarded as a standard for safe medication administration practices, “the five rights” are intended to guide the process of the medication administration programs by providing standard procedures for administration of (1) the right drug at (2) the right strength to (3) the right patient via (4) the right route at (5) the right time. In this way, it is believed that competent and safe care is reinforced by “the five rights”, which are aimed at ensuring the proper implementation and information communication. Although the focus of the five rights is on the performance of individuals, these individual efforts culminate in the entire process of delivering the drug to the patient in accordance with the doctor's instructions, which includes the efforts of a number of professionals from various disciplines; wherein the responsibility for accurate drug administration lies with multiple individuals and their part in reliable systems.

However, although these criteria mark the goals of safe medication practice, many errors, including lethal mistakes, have occurred even when healthcare professionals were confident that they had verified these “rights.” Clearly the number of steps that are involved in the process of medication administration creates, at each step, the ability to introduce errors. In this respect, although the program has benefited from the experience and involvement of the individuals served, their families, service providers and professional organizations to implement uniform, statewide standards that undergo continuous evaluation and improvement to support “the five rights,” such programs lack the continuity of care between all stakeholders in the process, particularly with the increased risk that is incurred with every step required in integrating the platforms of all of these stakeholders.

Accordingly, after decades of the existing methodologies, there remains significant need for additional tools and systems that address these concerns and reduce the number of potential errors that could be introduced, more sufficiently adhering to the five rights while utilizing the multiple platforms needed for medical administration programs for community residential programs.

### SUMMARY OF THE INVENTION

Accordingly, the present invention is directed to novel unified interfaces that are suitable for reducing risk of errors introduced into multi-step and multi-center medical programs through the unification of the multiple platforms of each center. In particular, the present invention provides methods, tools, and systems that unify at least three centers, e.g., of medication administration programs, reducing the number of potential errors that could be introduced, more sufficiently adhering to the five rights while utilizing and unifying the multiple platforms, for example, as needed for medical administration programs for community residential programs.

As such, one aspect of the invention provides a medical platform unification tool comprising a unification interface suitable for facilitating omni-directional communication and control (ODCC) between three or more medical administration centers, through a central unified database. The medical administration centers are selected from the group consisting of a medical practitioner administration center, a pharmacy administration center, a patient administration center, an insurance administration center, and a caretaker administration center. Moreover, each of the centers are capable of accessing the unification interface to receive information, modify information, add to the information of the central unified database, or take action on information stored on the central unified database using the unification interface.

Another aspect of the present invention provides a medical platform unification system comprising a medical platform unification tool of the present invention, comprising a unification interface suitable for facilitating omni-directional communication and control (ODCC) between three or more medical administration centers, through a central unified database; and one or more devices capable of interfacing with said unification interface.

In another aspect, the present invention provides a method of medical platform unification. The method comprises the steps of: collection of medical information for storage in the central unified database; organizing information into a unified viewing platform that presents information for each medical administration center; and interfacing one medical administration center with at least two different medical administration centers by providing access to each medical administration center to the central unified database, facilitating omni-directional communication and control (ODCC),

such that omni-directional communication and control (ODCC) between three or more medical administration centers, through a central unified database, is facilitated.

### BRIEF DESCRIPTION OF THE DRAWINGS

Advantages of the present apparatus will be apparent from the following detailed description, which description should be considered in combination with the accompanying drawings, which are not intended limit the scope of the invention in any way.

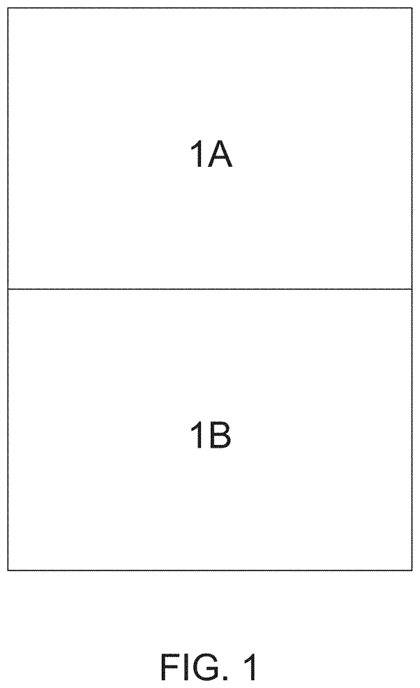

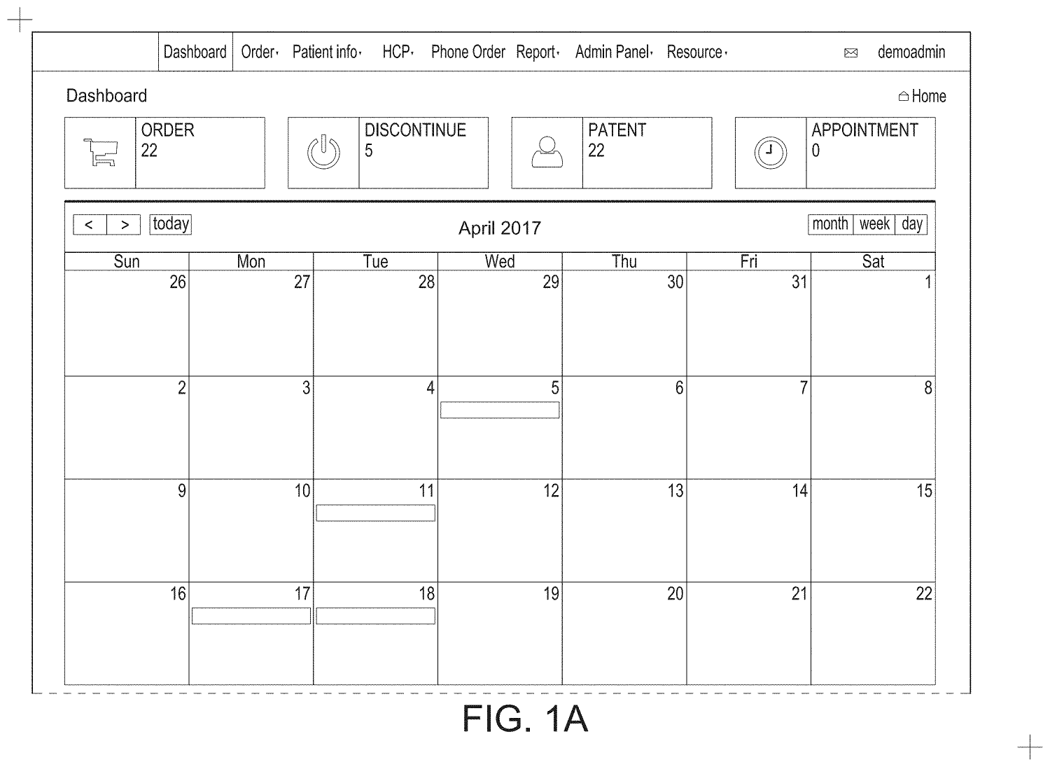

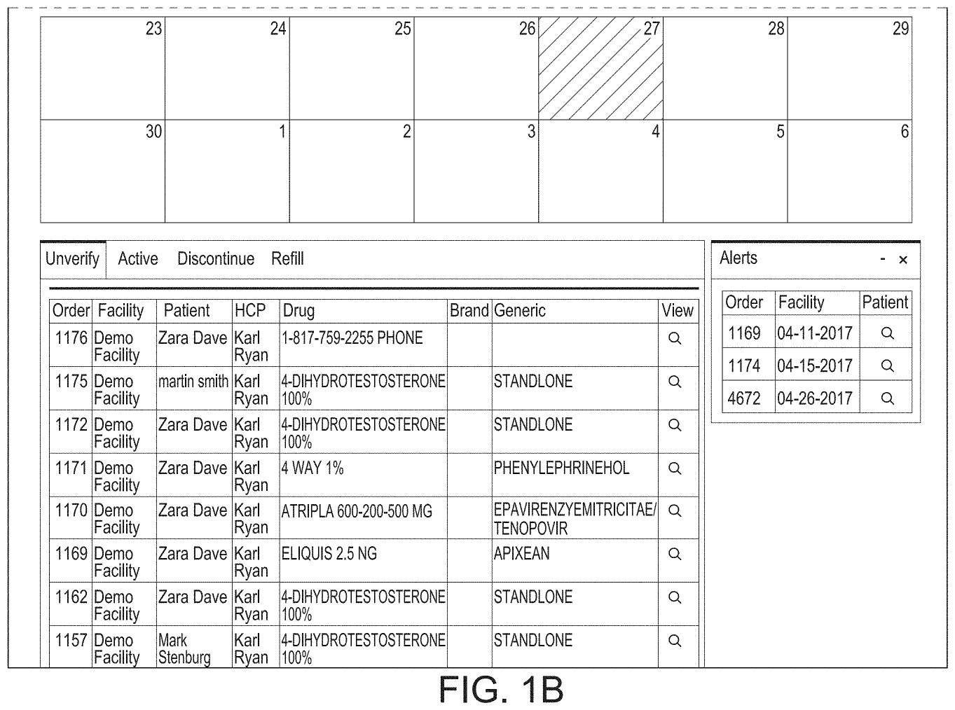

The attached FIG. 1, shown by the relation of FIG. 1A to FIG. 1B, and the combination of FIG. 1A with FIG. 1B depicts an example of a dashboard for certain particular embodiments of the unification interface of the medical platform unification tool of the present invention.

### DETAILED DESCRIPTION OF THE INVENTION

For decades, the multi-center medical programs, such as medication administration programs, have made use of separate systems of monitoring the multiple steps of the multiple centers; increasing the risk of introduction of errors into the process despite attempted compliance with the “five rights.” The present invention provides a safer, and more convenient solution to these problems through the creation of a unified interface. As such, the unification of the multiple centers through a central unified database reduces the potential introduction of errors. The unification of these multi-center medical programs has wide market appeal, especially from those in the industry of state regulated healthcare, which must strictly adhere to the state guidelines for multiple steps, and have been relegated to independent manual recordation and subsequent communication to the remaining centers. Through unification, the methods, tools, and systems of the invention reduce the overall number of vulnerable steps between prescription and administration.

Accordingly, the present invention is directed to novel unified interfaces that are suitable for reducing risk of errors introduced into multi-step and multi-center medical programs through the unification of the multiple platforms of each center. In particular, the present invention provides methods, tools, and systems that unify at least three centers, e.g., of medication administration programs, reducing the number of potential errors that could be introduced, more sufficiently adhering to the five rights while utilizing and unifying the multiple platforms, for example, as needed for medical administration programs for community residential programs.

Moreover, although the MAP program is an example of the utility of the unified interfaces detailed herein, such interfaces may find use in any technological areas that would find beneficial utility in unifying multi-center platforms.

The present invention, including medical platform unification tools, unification interfaces, methods, and systems will be described with reference to the following definitions that, for convenience, are set forth below. Unless otherwise specified, the below terms used herein are defined as follows:

### I. Definitions

As used herein, the term “a,” “an,” “the” and similar terms used in the context of the present invention (especially in the context of the claims) are to be construed to cover both the singular and plural unless otherwise indicated herein or clearly contradicted by the context.

The term “automatically” is used herein to describe a process that is automated or semi-automated. Automated processes do not contain steps that require a human operator to perform any steps. Semi-automated processes contain one or more steps that require a human operator; however, differ from manual processes by containing at least one step that does not require a human operator.

The term “communication” describes a type of interfacing directed to the act of accessing information or data from the central unified database for use within the functional utilities of the unification interface, and in certain embodiments, includes simple passive communication achieved by medical information being accessed for review. In particular embodiments, at least three of the medical administration centers are in communication through the unified database.

The language “consumer facing (CF) interface” is a user interface that is designed for use by a consumer of goods. In particular, such interface is designed for use by the consumer of the goods as opposed to the seller goods that would use this interface to monitor product for sale. More specifically, it is for use by the end purchaser, or agents thereof, e.g., caretakers or medical practitioners. As such, in certain embodiments of the invention, the interface is consumer facing only.

The term “control” as used herein in the language “omni-directional communication and control” describes a type of interfacing directed to the act of modifying the data or information on the central unified database, e.g., adding data, removing data, or editing data, i.e., using the unified interface; resulting in the information being modified for all medical administration centers. In particular embodiments, at least three of the medical administration centers control the unified databased information.

The term “expiration” is used herein to describe a date with which a product is labeled (or implicitly understood to be characteristic of such product) that beyond which the product has exceeded the anticipated shelf life. In certain embodiments, this date represents the date after which the product should no longer be used, either by operation of law or well-known convention. In particular embodiments, the expiration date is a date up until which the manufacturer guarantees the full potency and safety of the product. In specific embodiments, this date is absolutely definable, or definite, and is described herein as a definable expiration date.

The language “expiration data” is used herein to describe a combination of expiration date and product identification information. For clarity, expiration date would be considered a component of expiration data.

The term “HIPAA” is art-recognized, and is used herein as an abbreviation for the federal Health Insurance Portability and Accountability Act of 1996, a statute whose primary goal is to make it easier for people to keep health insurance, protect the confidentiality and security of healthcare information, and help the healthcare industry control administrative costs. In particular embodiments of the present invention, the communication and control of information within the unification interface is HIPAA compliant with respect to their ability to maintain the confidentiality and security of healthcare information.

The term “information” and “medical information” have been used herein interchangeably, and describe all information related to the medical administration centers.

The term “interface” is art-recognized, and is used herein to describe a shared boundary across which two or more separate interfacing components are enabled to exchange information, which can be, for example, between: software components, computer hardware components, peripheral device components, humans and combinations of thereof. Moreover, the operation of these separate components across the boundary, as in the interaction of the medical administration centers through the unified database, is referred to herein as “interfacing.” In particular embodiments, such interfacing may include communication with or control of the unified database. In certain embodiments, the interfacing may be bi-directional. In other embodiments, the interfacing may be uni-directional. In specific embodiments, the term “interface” may be a user interface, e.g., a graphic user interface.

The language “machine-readable medium” is art-recognized, and describes a medium capable of storing data in a format readable by a mechanical device (rather than by a human). Examples of machine-readable media include magnetic media such as magnetic disks, cards, tapes, and drums, punched cards and paper tapes, optical disks, barcodes, magnetic ink characters, and solid state devices such as flash-based, SSD, etc. Machine-readable medium of the present invention are non-transitory, and therefore do not include signals per se, i.e., are directed only to hardware storage medium. Common machine-readable technologies include magnetic recording, processing waveforms, and barcodes. In particular embodiments, the machine-readable device is a solid state device. Optical character recognition (OCR) can be used to enable machines to read information available to humans. Any information retrievable by any form of energy can be machine-readable. Moreover, any data stored on a machine-readable medium may be transferred by streaming over a network. In a particular embodiment, the machine-readable medium is a network server disk, e.g., an internet server disk, e.g., a disk array. In specific embodiments, the machine-readable medium is more than one network server disks.

The term “medical product” is used herein to describe any medicinal product or good with a well-characterized or definable expiration date, e.g., medicines (such as pills, ointments, syringes, etc.) as well as devices.

The language “notification action” is used herein to describe an action taken (such as an interface alert, email, text message, or the like) that notifies one or more groups of users, e.g., one or more medical administration centers, upon the occurrence of a certain event. In particular embodiments of the present invention, the notification may be based on the expiration differential indicating the expiration of a medical product.

The term “omni-directional” as used herein in the language “omni-directional communication and control” describes the universal ability for the same and equal communication and control of a database, e.g., of a unified database, by each member that is capable of accessing the database.

The language “patient database” is art-recognized, and is used herein to describe a database of patient and patient-specific prescription information. For example, in one embodiment, the patient database is based on pharmacy database software, e.g., Suite Rx, QS1, or Rx30.

The term “storing” is art-recognized, and is used herein to describe the act of saving data on a machine readable medium in a manner that such data is subsequently retrievable on that machine readable medium.

### II. Methods of Medical Platform Unification of the Invention

Accordingly, the present invention provides a method of medical platform unification comprising the steps of:

collection of medical information for storage in the central unified database;

organizing information into a unified viewing platform that presents information for each medical administration center; and

interfacing one medical administration center with at least two different medical administration centers by providing access to each medical administration center to the central unified database, facilitating omni-directional communication and control (ODCC),

such that omni-directional communication and control (ODCC) between three or more medical administration centers (e.g., 4 or more medical administration centers, e.g., 5 or more medical administration centers, e.g., 6 or more medical administration centers) through a central unified database, is facilitated (e.g., to enhance safety in a medication administration program). In certain embodiments, the central unified database may be stored on a second machine-readable medium.

In certain embodiments of the invention, the method of medical platform unification is useful for medication administration to assisted-residents in residential group care.

The omni-directional communication and control (ODCC) between the medical administration centers, through a central unified database, affords each medical center of the multi-center medical platform, e.g., each medical center of medication administration program (MAP), a means to interface with the each other, and in which each are capable of accessing and modifying/storing data in the unified database. In particular, no prior methods, tools or systems exist that have brought these medical administration centers into unification.

In certain embodiments of the methods of the invention, the medical administration centers are medication administration program (MAP) centers.

In certain embodiments of the invention, the method of medical platform unification comprises authentication of the medical administration center, i.e., for access by the medical administration center.

In certain embodiments of the methods of the invention, the step of collection of medical information for storage in the central unified database is automated, e.g., using an import tool. In certain alternative embodiments of the methods of the invention, the step of collection of medical information for storage in the central unified database is manual.

In certain embodiments of the methods of the invention, the step of collection occurs through the unified interface via entry by the medical administration centers, e.g., manually, automated, or a combination thereof.

In certain embodiments of the methods of the invention, the step of collection occurs through the unified interface via entry by an administrator of the unified interface, e.g., manually, automated, or a combination thereof.

A. Medical Practitioner Administration Center

The medical practitioner administration center, which may also be referred to as the healthcare provider (HCP), is charged with diagnosis of the medical conditions of a member of the patient administration center, e.g., a patient, and prescription of medical products thereto as described herein, e.g., drugs or devices. Examples of members of the medical practitioner administration center includes, for example, doctors, medical offices, hospitals, etc.

In certain embodiments of the invention, the central unified interface (see, for example the attached Figure), collects and stores information accessible and capable of modification related to the medical practitioner administration center such as: details related to the HCP, e.g., HCP listings, HCP specialty, or HCP visit information (including patient and medication update). In particular embodiments, HCP database software, such as a patient database, may be used to derive the information collected.

B. Pharmacy Administration Center

The pharmacy administration center is charged with receiving and filling a prescription ordered by the medical practitioner administration center. Examples of members of the pharmacy administration center include, for example, pharmacies, e.g., stand-alone or hospital associated.

In certain embodiments of the invention, the central unified interface (see, for example the attached Figure), collects and stores information accessible and capable of modification related to the pharmacy administration center such as: pharmacy listings, medical product listings, medical product order status (e.g., ordered, reordered, on order, needs ordering, needs ordering on date certain, etc.), medical product expiration status, pharmacy related patient information, and/or pharmacy related drug information as associated to a specific patient and based upon prescription. In particular embodiments, pharmacy database software, such as a patient database (e.g., Suite Rx, QS1, or Rx30), may be used to derive the information collected.

In certain embodiments, the stored/accessible information satisfies some or all of the prescription requirements of the Federal Food Drug & Cosmetic Act, for example, indicating one or more of the following:
i. the name of the individual;
ii. the name of the controlled substance (medication) including the IC (i.e., interchange) name, if any, e.g., the IC name of the medication may be listed when the pharmacy dispenses a generic substitution or a less expensive brand name drug product for the prescribed medication;
iii. the strength and amount;
iv. the frequency;
v. the route of administration;
vi. the name of the prescribing health care provider;
vii. directions for use;
viii. cautionary statements, if any;
ix. total quantity of medication dispensed (e.g., if the medication was dispensed as tablets or capsules, the number of same in the container);
x. the date of filling;
xi. the lot number;
xii. the pharmacy name, address, telephone number and serial/prescription number;
xiii. the expiration date; and
iv. the filling pharmacist's initials.

C. Patient Administration Center

The patient administration center is defined as the patient and any authorized agents to act on behalf of the patient, e.g., family or guardians. The patient administration center information may be collected from any of the medical administration centers, which is aggregated on the central unified database. In certain embodiments of the invention, the central unified interface (see, for example the attached Figure), collects and stores information accessible and capable of modification related to the patient administration center such as: identification, medical product, e.g., drugs, and prescription information. In particular embodiments, such information may include any one of the following:
1. individual's name;
2. name and strength of medication;
3. directions for usage (e.g., clearly stated; including specific doses, dosing times, and dosing route);
4. prescribing health care provider's name;
5. date of dispensing;
6. any necessary cautionary statements (e.g., take with food.); and
7. amount of medication, or any combination thereof.

D. Caretaker Administration Center

The caretaker administration center is charged with giving care to a patient, based upon instructions presented from the medical administration center, pharmacy administration center, and general nursing principals. Such caretaker administration centers are guided by both insurance administration center requirements as well as government administration center requirements. Examples of members of the caretaker administration center includes, for example, community residential programs and facilities that ensure the safety of individuals living within them, such as group homes.

In certain embodiments of the invention, the central unified interface (see, for example the attached Figure), collects and stores information accessible and capable of modification related to the caretaker administration center such as: administration schedules, reports, inventory, medical product order status (e.g., ordered, reordered, on order, needs ordering, needs ordering on date certain, etc.), medical product expiration status, patient information, and/or drug information as associated to a specific patient and based upon prescription.

E. Insurance Administration Center

In certain embodiments, the insurance administration center is a medical administration center of the present invention, and is charged with overseeing the insurance, and reimbursable/nonreimbursable aspects of treatment and care for a patient.

In certain embodiments of the invention, the central unified interface collects and stores information accessible and capable of modification related to the insurance administration center such as: list of insurers, insurance plan coverage, insurance plan type, and/or insurance plan limitations.

F. Government Administration Center

In certain embodiments, one of the medical administration centers is a government administration center, e.g., state or federal, and is charged with providing government oversight of treatment and care for a patient, e.g., a government case work, as well as insuring proper legal compliance with statutes and regulations. In particular embodiments, the government administration center may be the state government or the federal government, e.g., the FDA.

In certain embodiments of the invention, the method of medical platform unification further comprises the step of tracking the product. In particular embodiments, the step of tracking the product further comprises the step of storing product status, count, and location. In particular embodiments, tracking information may additionally include information related to the medical product and the patient.

In certain embodiments of the invention, the method of medical platform unification further comprises the step of tracking the patient. In particular embodiments, the step of tracking the patient further comprises the step of storing patient status, treatment status, e.g., delivery of medication, and treatment results.

In certain embodiments of the methods of the invention, the method further comprises the step of creating and storing an electronic record that serves as an electronic medical record (EMR). In particular embodiments, the record identifies a person making the change, e.g., authorized or authenticated person.

In certain embodiments of the invention, the method of medical platform unification is HIPAA compliant. In particular embodiments the method of medical platform unification is in compliance with medication administration programs (MAP), e.g., compliant with the Massachusetts Medication Administration Program policies.

In certain embodiments of the invention, the method of medical platform unification presents a calendar of chronological relevant dates for each medical administration center, e.g., on a dashboard.

In certain embodiments of the invention, the method of medical platform unification affords the ability to order or reorder a medical product, e.g., add order, view, order, refill history, medical record.

In certain embodiments of the invention, the method of medical platform unification stores and presents information selected from the group consisting of patient information, medical practitioner information, pharmacy information, caretaker information, insurance information, and any combination thereof.

In certain embodiments of the invention, the method of medical platform unification provides electronic communication between medical administration centers through email or instant message.

In certain embodiments of the invention, the method of medical platform unification further comprises an expiration monitoring control module capable of capturing expiration data, storing expiration data, analyzing expiration data to determine expiration status of a medical product, and generating action based on the expiration status of the medical product.

In certain embodiments of the invention further comprising an expiration monitoring control module, the method of medical platform unification further comprises the steps of:

capturing the expiration data of a medical product;

storing the captured expiration data of the medical product (e.g., may be stored on a second machine readable medium);

analyzing said stored expiration data of the medical product to determine an expiration status of the medical product, which is established from the expiration differential produced by comparison of the current date to the expiration date of the medical product; and

generating a notification action based on the expiration differential indicating the expiration of a medical product, e.g., within a defined period of time, such that expiration of the medical product is monitored.

In certain embodiments of the methods of the invention, the notification action is an alert on an expiration monitoring control module interface, e.g., on the dashboard.

In certain embodiments of the methods of the invention with an expiration monitoring control module, the notification action is an email.

In certain embodiments of the methods of the invention with an expiration monitoring control module, the notification action is a text message.

In certain embodiments of the methods of the invention with an expiration monitoring control module, the notification action generates a reorder request, e.g., automatically reorders.

In certain embodiments of the methods of the invention with an expiration monitoring control module, capturing the expiration data of a product may occur through manual data entry, scanning of product codes, e.g., UPC, or scanning image/text conversion.

In certain embodiments of the methods of the invention with an expiration monitoring control module, an interface presents a calendar of expiration dates for each product, e.g., on the dashboard.

In certain embodiments of the methods of the invention with an expiration monitoring control module, an interface affords the ability to reorder the product.

### III. Medical Platform Unification Tool of the Invention

The methods of the present invention are useful as instructions stored on a machine-readable medium for execution by a processor to perform the method. In certain embodiments, the methods and tools of the present invention also make use and/or comprise a processor. Accordingly, any methods of the present invention, alone or in combination with other methods (such as those described herein or elsewhere) may be stored on a machine-readable medium for execution by a processor to perform the method. Such a composition comprises a medical platform unification tool of the invention, e.g., a unification interface.

In this respect, the methods of medical platform unification provide for facilitating omni-directional communication and control (ODCC) between three or more medical administration centers (e.g., 4 or more medical administration centers, e.g., 5 or more medical administration centers, e.g., 6 or more medical administration centers), through a central unified database. In certain embodiments, the methods provide for interfacing with the medical administration centers through the unified database, e.g., on a server. The interfacing tools of the present invention are user interfaces for providing medical platform unification, e.g., graphical user interfaces (GUI), which are user-facing for each of the medical administration centers interacting with other medical administration centers to transmit information, collect information, as well as act on that information through combination, analysis, and ultimately providing diagnostic analysis through medical platform unification.

One embodiment of the invention provides a medical platform unification tool comprising a unification interface suitable for facilitating omni-directional communication and control (ODCC) between three or more medical administration centers (e.g., 4 or more medical administration centers, e.g., 5 or more medical administration centers, e.g., 6 or more medical administration centers), through a central unified database,

wherein the medical administration centers are selected from the group consisting of a medical practitioner administration center, a pharmacy administration center, a patient (e.g., accessible by authorized family) administration center, an insurance administration center, and a caretaker (e.g., facility) administration center, and

wherein each of the centers are capable of accessing the unification interface to receive information, modify information, add to the information of the central unified database, or take action on information stored on the central unified database using the unification interface.

In certain embodiments of the medical platform unification tool, the unification interface comprises a machine readable medium having instructions stored thereon for execution by a processor to perform a method comprising the steps of:

collection of medical information for storage in the central unified database;

organizing information into a unified viewing platform that presents information for each medical administration center; and

interfacing one medical administration center with at least two different medical administration centers by providing access to each medical administration center to the central unified database, and facilitating omni-directional communication and control (ODCC),

such that omni-directional communication and control (ODCC) between three or more medical administration centers (e.g., 4 or more medical administration centers, e.g., 5 or more medical administration centers, e.g., 6 or more medical administration centers), through a central unified database, is facilitated (e.g., to enhance safety in the medication administration program).

In certain embodiments of the invention, the medical platform unification tool is useful for medication administration to assisted-residents in residential group care.

In certain embodiments of the medical platform unification tool, the medical administration centers are medication administration program (MAP) centers.

In certain embodiments of the medical platform unification tool, the unification interface comprises authentication of the medical administration center, i.e., for access by the medical administration center.

In certain embodiments of the medical platform unification tool, the step of collection of medical information for storage in the central unified database is automated, e.g., using an import tool. In certain alternative embodiments of the methods of the invention, the step of collection of medical information for storage in the central unified database is manual.

In certain embodiments of the medical platform unification tool, the step of collection occurs through the unified interface via entry by the medical administration centers, e.g., manually, automated, or a combination thereof.

In certain embodiments of the medical platform unification tool, the step of collection occurs through the unified interface via entry by an administrator of the unified interface, e.g., manually, automated, or a combination thereof.

In certain embodiments of the medical platform unification tool, the unification interface presents a calendar of chronological relevant dates for each medical administration center, e.g., on a dashboard.

In certain embodiments of the medical platform unification tool, the unification interface affords the ability to order or reorder a medical product, e.g., add order, view, order, refill history, medical record.

In certain embodiments of the medical platform unification tool, the unification interface stores and presents information selected from the group consisting of patient information, medical practitioner information (e.g., healthcare provider (HCP) information, e.g., HCP listings, HCP specialty, or HCP visit information (including patient and medication update)), pharmacy information (e.g., drugs listings), caretaker (e.g., facility) information (e.g., reports), insurance information, and any combination thereof.

In certain embodiments of the medical platform unification tool, the unification interface serves as an electronic medical record (e.g., identifies person making the change by authorization/authentication).

In certain embodiments of the medical platform unification tool, the unification interface is HIPAA compliant.

In certain embodiments of the medical platform unification tool, the unification interface provides electronic communication between medical administration centers through email or instant message.

In certain embodiments, the medical platform unification tool further comprises an expiration monitoring control (EMC) module capable of capturing expiration data, storing expiration data, analyzing expiration data to determine expiration status of a medical product, and generating action based on the expiration status of the medical product. In certain embodiments, the EMC module comprises instructions stored on the machine readable medium for execution by a processor to perform a method comprising the steps of:

capturing the expiration data of a medical product;

storing the captured expiration data of the medical product (e.g., may be stored on a second machine readable medium);

analyzing said stored expiration data of the medical product to determine an expiration status of the medical product, which is established from the expiration differential produced by comparison of the current date to the expiration date of the medical product; and

generating a notification action based on the expiration differential indicating the expiration of a medical product, e.g., within a defined period of time, such that expiration of the medical product is monitored.

In certain embodiments, the medical platform unification tool further comprises an EMC module interface.

In certain embodiments, the medical platform unification tool further comprises an EMC module interface, wherein the interface is a consumer facing (CF) interface.

In certain embodiments of the medical platform unification tool, the notification action is an alert on the EMC module interface, e.g., on the dashboard.

In certain embodiments of the medical platform unification tool further comprising an expiration monitoring control (EMC) module, the notification action is an email.

In certain embodiments of the medical platform unification tool further comprising an expiration monitoring control (EMC) module, the notification action is a text message.

In certain embodiments of the medical platform unification tool further comprising an expiration monitoring control (EMC) module, the notification action generates a reorder request, e.g., automatically reorders.

In certain embodiments of the medical platform unification tool further comprising an expiration monitoring control (EMC) module, capturing the expiration data of a product may occur through manual data entry, scanning of product codes, e.g., UPC, or scanning image/text conversion.

In certain embodiments of the medical platform unification tool further comprising an expiration monitoring control (EMC) module, the interface presents a calendar of expiration dates for each product, e.g., on the dashboard.

In certain embodiments of the medical platform unification tool further comprising an expiration monitoring control (EMC) module, the interface affords the ability to reorder the product.

In certain embodiments of the medical platform unification tool, method of medical platform unification further comprises the step of tracking the product. In particular embodiments, the step of tracking the product further comprises the step of storing product status, count, and location. In particular embodiments, tracking information may additionally include information related to the medical product and the patient.

In certain embodiments of the medical platform unification tool, the method of medical platform unification further comprises the step of tracking the patient. In particular embodiments, the step of tracking the patient further comprises the step of storing patient status, treatment status, e.g., delivery of medication, and treatment results.

In certain embodiments of the medical platform unification tool, the machine-readable medium is selected from the group consisting of magnetic media, punched cards, paper tapes, optical disks, barcodes, magnetic ink characters, and solid state devices.

### IV. Systems of the Invention

The medical platform unification tool of the present invention, described herein, may be incorporated into systems, which may also comprise one or more devices.

As such, another embodiment of the present invention provides a medical platform unification system comprising

an medical platform unification tool described herein, comprising a unification interface suitable for facilitating omni-directional communication and control (ODCC) between three or more medical administration centers, through a central unified database; and

one or more devices capable of interfacing with said unification interface.

In certain embodiments of the invention, the system further comprises a device to capture expiration data of a medical product.

A. Device Capable Of Interfacing With Unification Interface

In certain embodiments, the systems of the present invention comprise a device that is capable of interfacing with the unification interface of the present invention. Such devices include one or more wireless devices (e.g., smartphones, tablets, and laptop computers), desktop computers, or suitably operationally associated monitors.

B. Device to Capture Expiration Data

In certain embodiments, the systems of the present invention comprise a device to capture the expiration data of a product. The device affords the ability to semi-automate or automate certain data entry aspects of the capture of expiration data of a product, e.g., for an expiration monitoring control (EMC) module, by capturing the expiration data of a product scanning of product codes, e.g., UPC, or scanning image/text conversion. Such devices include scanning devices with a primary utility of scanning information through image capture or text capture (e.g., including bar codes) that may or may not be operationally associated with a second device relied upon for interpretation of the image or text, or a device that has a secondary purpose that is capable of capturing image and text, e.g., wireless devices such as smartphones and tablets.

### V. Design Aspects of the Invention

Independent of the utility related to unification interface of the medical platform unification tool of the present invention, the ornamental appearance of any novel design provided herein is intended to be part of this invention, for example, the dashboard design in the attached Figure, which may form an independent or combined ornamental appearance of the unification interface described herein.

Accordingly, one embodiment of the present invention provide an ornamental design for a unification interface as shown and described.

### Exemplification

Having thus described the invention in general terms, reference will now be made to the accompanying drawings of exemplary embodiments, which are not necessarily drawn to scale, and which are not intended to be limiting in any way.

In this respect, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The attached Figure depicts an example of a dashboard for certain particular embodiments of the unification interface of the medical platform unification tool of the present invention. Certain drop-down menus are presented at the top of the screen, including: Dashboard, Order, Patient Info, HCP, Report, and Admin Panel. Email notifications are presented in the upper right hand of the page, e.g., adjacent to the username.

The Order drop-down list contains sub-selections of: Add Order, View Order, Refill History, and Medical Record.

The Patient Info drop-down list contains sub-selections of: Add Patient and View Patient.

The HCP drop-down list contains sub-selections of: Add HCP, View HCP, HCP Specialty, HCP Visit, HCP Visit Patient Update, and HCP Visit Medication Update.

The Report drop-down list contains sub-selections of: Medication and Treatment.

The Admin Panel drop-down list contains sub-selections of: User Registration, User Allocate Facility, Facility, Pharmacy Store, Drug List, Allergy, Amount, Route, Purpose, Frequency, Special Instruction, Insurance, Insurance Priority, and Medical record category.

Further, the unification interface presents a calendar of chronological relevant dates for each medical administration center.

In addition, certain higher level easy access functions are presented in larger color coded boxes along with the number of pending actions for the respective box category, including Order (e.g., in blue), Discontinue (e.g., in red), Patient (e.g., in green), and Appointment (e.g., in yellow).

Additional windows presenting data from the central unified database may be sorted based on any categorical criteria (such as order number, patient name, facility, drug, or brand) and presented on the Dashboard, with multiple layers, e.g., based on status.

An alert panel is also presented on the Dashboard. The particular alert depicted in the interface corresponds to the notification action of the EMC module, and is presented with information related to order number, expiration date, and a link for viewing additional information. Additional information may be presented in the alert, or held in the material that is linked therewith. In particular embodiments, alerts may be color coded, e.g., for urgency.

### Incorporation By Reference

The entire contents of all patents, published patent applications and other references cited herein are hereby expressly incorporated herein in their entireties by reference.

### Equivalents

Those skilled in the art will recognize, or be able to ascertain using no more than routine experimentation, numerous equivalents to the specific procedures described herein. Such equivalents were considered to be within the scope of this invention and are covered by the following claims. Moreover, any numerical or alphabetical ranges provided herein are intended to include both the upper and lower value of those ranges. In addition, any listing or grouping is intended, at least in one embodiment, to represent a shorthand or convenient manner of listing independent embodiments; as such, each member of the list should be considered a separate embodiment.

What is claimed is:

1. A medical platform unification tool comprising a unification interface suitable for facilitating omni-directional communication and control (ODCC) between three or more medical administration centers, through a central unified database,
wherein the medical administration centers are selected from the group consisting of a medical practitioner administration center, a pharmacy administration center, a patient administration center, an insurance administration center, and a caretaker administration center, and
wherein each of the centers are capable of accessing the unification interface to receive information, modify information, add to the information of the central unified database, or take action on information stored on the central unified database using the unification interface.

2. The medical platform unification tool of claim 1, wherein the unification interface comprises a machine readable medium having instructions stored thereon for execution by a processor to perform a method comprising the steps of:
collection of medical information for storage in the central unified database;
organizing information into a unified viewing platform that presents information for each medical administration center; and
interfacing one medical administration center with at least two different medical administration centers by providing access to each medical administration center to the central unified database, and facilitating omni-directional communication and control (ODCC),
such that omni-directional communication and control (ODCC) between three or more medical administration centers, through a central unified database, is facilitated.

3. The medical platform unification tool of claim 1 useful for medication administration to assisted-residents in residential group care.

4. The medical platform unification tool of claim 1, wherein the medical administration centers are medication administration program (MAP) centers.

5. The medical platform unification tool of claim 1, wherein accessing the unification interface comprises authentication of the medical administration center.

6. The medical platform unification tool of claim 1, wherein the step of collection of medical information for storage in the central unified database is automated.

7. The medical platform unification tool of claim 1, wherein the unification interface presents a calendar of chronological relevant dates for each medical administration center.

8. The medical platform unification tool of claim 1, wherein the unification interface affords the ability to order or reorder a medical product.

9. The medical platform unification tool of claim 1, wherein the unification interface stores and presents information selected from the group consisting of patient information, medical practitioner information, pharmacy information, caretaker information, insurance information, and any combination thereof.

10. The medical platform unification tool of claim 1, wherein the unification interface serves as an electronic medical record.

11. The medical platform unification tool of claim 1, wherein the unification interface is HIPAA compliant.

12. The medical platform unification tool of claim 1, wherein the unification interface provides electronic communication between medical administration centers through email or instant message.

13. The medical platform unification tool of claim 1 further comprising an expiration monitoring control (EMC) module capable of capturing expiration data, storing expiration data, analyzing expiration data to determine expiration status of a medical product, and generating action based on the expiration status of the medical product.

14. The medical platform unification tool of claim 13, wherein wherein the EMC module comprises instructions stored on the machine readable medium for execution by a processor to perform a method comprising the steps of:
capturing the expiration data of a medical product;
storing the captured expiration data of the medical product;
analyzing said stored expiration data of the medical product to determine an expiration status of the medical product, which is established from the expiration differential produced by comparison of the current date to the expiration date of the medical product; and
generating a notification action based on the expiration differential indicating the expiration of a medical product, e.g., within a defined period of time, such that expiration of the medical product is monitored.

15. The medical platform unification tool of claim 1, wherein the machine-readable medium is selected from the group consisting of magnetic media, punched cards, paper tapes, optical disks, barcodes, magnetic ink characters, and solid state devices.

16. A medical platform unification system comprising
a medical platform unification tool of claim 1, comprising a unification interface suitable for facilitating omni-directional communication and control (ODCC) between three or more medical administration centers, through a central unified database; and
one or more devices capable of interfacing with said unification interface.

17. The medical platform unification system of claim 16, further comprising a device to capture expiration data of a medical product.

18. A method of medical platform unification comprising the steps of:
collection of medical information for storage in the central unified database;
organizing information into a unified viewing platform that presents information for each medical administration center; and
interfacing one medical administration center with at least two different medical administration centers by providing access to each medical administration center to the central unified database, facilitating omni-directional communication and control (ODCC),
such that omni-directional communication and control (ODCC) between three or more medical administration centers, through a central unified database, is facilitated.

19. The method of claim 18 further comprising capturing expiration data, storing expiration data, analyzing expiration data to determine expiration status of a medical product, and generating action based on the expiration status of the medical product.

20. The method of claim 19, wherein the method comprises the steps of:
capturing the expiration data of a medical product;
storing the captured expiration data of the medical product;
analyzing said stored expiration data of the medical product to determine an expiration status of the medical product, which is established from the expiration differential produced by comparison of the current date to the expiration date of the medical product; and
generating a notification action based on the expiration differential indicating the expiration of a medical product,
such that expiration of the medical product is monitored.
